(19) United States
(12) Patent Application Publication
(10) Pub. No.: US 2024/0026394 A1
(43) Pub. Date: Jan. 25, 2024

(54) ENZYME BASED PRODUCTION OF SPECIALIZED PRO-RESOLVING MEDIATORS (SPMS) VIA DOCOSAHEXAENOIC ACID (DHA) AND EICOSAPENTAENOIC ACID (EPA)

(71) Applicant: Evonik Operations GmbH, Essen (DE)

(72) Inventors: Rima JABER, Frankfurt (DE); Katharina BERSE, Waltrop (DE); Eva Maria WITTMANN, Traunreut (DE)

(73) Assignee: Evonik Operations GmbH, Essen (DE)

(21) Appl. No.: 18/265,094

(22) PCT Filed: Nov. 19, 2021
(86) PCT No.: PCT/EP2021/082317

§ 371 Date: Jun. 2, 2023

(30) Foreign Application Priority Data
Dec. 4, 2020 (EP) 20211739.6

(51) Int. Cl. C12P 7/6434

(52) U.S. Cl. CPC C12P 7/6434

(57) ABSTRACT

The present invention refers to a method for producing hydroxylated fatty acids by oxidizing at least one unsaturated fatty acid by at least one lipoxygenase and thereafter reducing the obtained compound by at least one peroxidase and/or heating. Furthermore, the present invention refers to the compound obtained by said method.

### FIELD OF THE INVENTION

The present invention refers to a method for producing hydroxylated fatty acids by oxidizing at least one unsaturated fatty acid by at least one lipoxygenase and thereafter reducing the obtained compound by at least one peroxidase and/or heating. Furthermore, the present invention refers to the compound obtained by said method.

### BACKGROUND OF THE INVENTION

Prostaglandins play a key role in inflammation and the counterpart to prostaglandins are known as Specialized Pro-resolving Mediators (SPMs). The SPM's role in reducing inflammation has been discussed widely in literature. Inhibition of inflammatory responses has been shown in cell systems (i.e. in vitro) and in vivo, with a fundamental role in the maintenance of tissue homeostasis. For example, the omega-3 fatty acids docosahexaenoic acid (DHA) and eicosapentaenoic acid (EPA) are the precursors of D and E-series resolvins, respectively (Valdes A. M. et al. “Association of the resolving precursor 17-HDHA, but not D- or E-series resolvins, with heat pain sensitivity and osteoarthritis pain in humans”, Sci. Rep. 2017, 7(1), p. 10748).

Omega-3 may be used to resolve inflammatory exudates to produce structurally distinct families of signalling molecules namely resolvins, protectins and maresins, collectively termed SPM. However, the endogenous production of SPMs is insufficient to have the full required effect. Human beings usually try to compensate the lack of innate production of SPMs through nutrition. However, due to diets which include insufficient amounts of omega-3, or excess omega-6, he conversion of omega-3 in the body to different SPMs is slow and inefficient as omega-3 and omega-6 compete for the same conversion enzymes (Simopoulos, A. P. “An Increase in the Omega-6/Omega-3 Fatty Acid Ratio Increases the Risk for Obesity” Nutrients, 2016, 8(3), 128).

Therefore, since exogenous intake of SPMs is effective and can reduce inflammation in living things including human beinds, there is a need in the art for a method, which provides SPMs, in particular derived from DHA and EPA, suitable for ingestion with a sufficient yield.

### DESCRIPTION OF THE INVENTION

The inventors of the present invention surprisingly found that the problems above can be solved by the specific process of the present invention.

In particular, the object has been solved by a method according to an aspect of the present invention for producing hydroxylated fatty acids, comprising or consisting of the steps:
ii) oxidizing by at least one lipoxygenase of at least one unsaturated fatty acid to produce an oxidised compound, wherein the oxidizing is performed at temperatures of 5 to 10° C.;
iii) reducing the oxidised compound obtained in step ii) by
   iiia) at least one peroxidase, and/or
   iiib) heating, and
iv) thereafter adjusting the pH value to at most 4.5 to obtain at least one hydroxylated fatty acid.

The method according to any aspect of the present invention, may optionally comprise steps:
i) optionally saponification or hydrolyzation of at least one unsaturated fatty acid ester to obtain at least one unsaturated fatty acid; and/or
v) optionally purifying the compound obtained in step iv).

The term “saponification” as used herein refers to the reaction of a fat or oil with a metallic alkali to form soap. In the process of saponification, the metal alkali breaks the ester bond in the unsaturated fatty ester and releases the unsaturated fatty acid. In particular, saponification is the alkaline hydrolysis of the fatty acid esters. This reaction is catalysed by a strong acid or base. The mechanism of saponification is: (a) Nucleophilic attack by the hydroxide, (b) Leaving group removal and (c) Deprotonation. It would be within the common knowledge of a skilled person to carry out saponification of an unsaturated fatty acid ester to form an unsaturated fatty acid. In one example, the unsaturated fatty acid ester of step (i) according to any aspect of the present invention is brought into contact with at least one metal alkali. In particular, the metal alkali is in aqueous form. More in particular, the aqueous metal alkali may be selected from KOH and NaOH.

Hydrolyzation of at least one unsaturated fatty acid ester to obtain at least one unsaturated fatty acid may also be carried out by at least one lipase. Lipase is a subclass of the esterases, which is a hydrolase enzyme that can split esters into an acid and an alcohol in a chemical reaction with water called hydrolysis. Any lipase which can perform the hydrolyzation of an unsaturated fatty acid ester is suitable. Particularly suitable are lipases having the EC number EC 3.1.1.3-triacylglycerol lipase.

In one embodiment the at least one lipase is present in 0.01 to 5 wt.-% based on the total weight of the oil Omega-3 fatty acid. In another example, the lipase is present in 0.05 to 5, 0.1 to 5, 0.15 to 5, 0.2 to 5, 0.25 to 5, 0.3 to 5, 0.4 to 1, 0.5 to 5, 1 to 5, 1.5 to 5, 2 to 5, 2.5 to 5, 3 to 5, or 3.5 to 5 wt.-%. More in particular, the lipase is present in about 0.01, 0.5, 1, 1.5, 2, 2.5, 3, 3.5, 4, 4.5 or 5 wt.-% based on the total weight of the oil Omega-3 fatty acid.

Lipoxygenases are a family of (non-heme) iron containing enzymes that catalyzes the deoxygenation of PUFAs yielding hydroperoxyl derivatives including hydroperoxy-eicosatetraenoic acids (HPETEs). Any lipoxygenase which can perform the oxidation of an unsaturated fatty acid is suitable. In particular, microbial lipoxygenases may be derived from, e.g., Saccharomyces cerevisiae, Thermoactinomyces vulgaris, Fusarium oxysporum, Fusarium proliferatum, Thermomyces lanuginosus, Pyricularia oryzae, and strains of Geotrichum. The preparation of a lipoxygenase derived from Gaeumannomyces graminis is described in Examples 3-4 of WO 02/20730. The expression in Aspergillus oryzae of a lipoxygenase derived from Magnaporthe salvinii is described in Example 2 of WO 02/086114, and this enzyme can be purified using standard methods, e.g., as described in Example 4 of WO 02/20730. Lipoxygenases may also be extracted from plant seeds, such as soybean, pea, chickpea, and kidney bean. Alternatively, lipoxygenase may be obtained from mammalian cells, e.g., rabbit reticulocytes. More in particular, the lipoxygenase used according to any aspect of the present invention may be obtained from soy, like soy flour, soy beans or soy meal, a supernatant or mixtures thereof. Even more in particular, the lipoxygenases from soybeans: EC 1.13.11.12 Linoleate:oxygen oxidoreductase may be used according to any aspect of the present invention.

In one embodiment the at least one lipoxygenase is present in 0.01 to 5 wt.-% based on the total weight of the at least one unsaturated fatty acid ester. In another example, the lipoxygenase is present in 0.05 to 5, 0.1 to 5, 0.15 to 5, 0.2 to 5, 0.25 to 5, 0.3 to 5, 0.4 to 1, 0.5 to 5, 1 to 5, 1.5 to 5, 2 to 5, 2.5 to 5, 3 to 5, or 3.5 to 5 wt.-%. More in particular, the lipase is present in about 0.01, 0.5, 1, 1.5, 2, 2.5, 3, 3.5, 4, 4.5 or 5 wt.-% based on the total weight of the oil Omega-3 fatty acid.

In the present method according to any aspect of the present invention, the at least one unsaturated fatty acid can be a single unsaturated fatty acid or a mixture of several different unsaturated fatty acids. In preferred embodiments, a mixture of several different unsaturated fatty acids is used. This is often due to the source of the unsaturated fatty acids, which can for example be a natural product, comprising several kinds of unsaturated fatty acids. For example, the at least one unsaturated fatty acid can be obtained from commercially available fish oil.

In one embodiment the at least one unsaturated fatty acid is at least one omega-3 fatty acid, preferably selected from docosahexaenoic acid (DHA), eicosatetraenoic acid, eicosapentaenoic acid (EPA) or a mixture thereof, more preferably selected from docosahexaenoic acid (DHA), eicosapentaenoic acid (EPA) or a mixture thereof.

In the following, the exemplary reaction scheme according to any aspect of the present invention for DHA is shown:

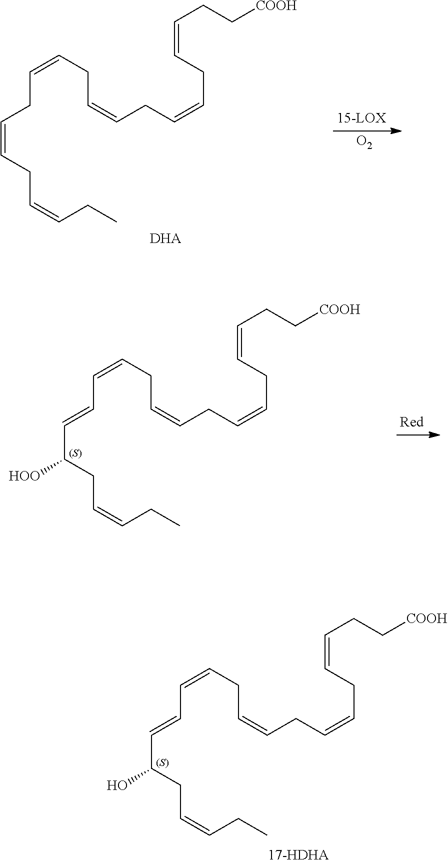

15-LOX=Lipoxygenase (Oxidation step)
O₂=Oxygen is added to process
Red=Reduction

The method according to any aspect of the present invention requires that the starting compound in the oxidation step ii) is at least one unsaturated fatty acid. If the starting compound should be an ester etc. thereof, the compound has to be brought into the form of at least one unsaturated fatty acid.

In one embodiment this can be done by saponification or hydrolysation, preferably by at least one lipase, of at least one unsaturated fatty acid ester.

The oxidation of the at least one unsaturated fatty acid takes place by at least one lipoxygenase, preferably in the presence of a buffer. In one embodiment the buffer is an aqueous buffer comprising Na₂CO₃/NaHCO₃. The mixture comprising the buffer preferably has a pH value of 9 to 10, more preferably 9.8. The oxidation step can be performed under stirring and/or at temperatures of 5 to 25° C., preferably 5 to 10° C., more preferably 5° C. In particular, the oxidation step may be carried out within a temperature range of 5 to 15° C., or 5 to 10° C. In another example, the oxidation step may be carried out at a temperature of about 5, 6, 7, 8, 9, or 10° C. It was an unexpected result that the lipoxygenase was found to be the most efficient, producing the highest yield at such low temperatures (i.e. 5-10° C.). Further, when oxidation was carried out according to any aspect of the present invention at the temperature between 5-10° C., lesser by-products were also produced therefore resulting in more of the desired product being produced. Prior art such as Tu, H-A. T et. al (2018) New Biotechnology, 41: 25-33, shows that lipoxygenases, in particular lipoxygenases from soy flour may be best used at room temperature.

In one embodiment the pH value is kept in a constant pH value range of the desired value+/−0.2 throughout the whole oxidation step.

In the context of the present invention, the term “about” denotes an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific deviation for a numerical value for a given technical effect will depend on the nature of the technical effect.

In addition to the at least one lipoxygenase in the oxidation step, at least one co-factor can be present, preferably selected from ammonium ferric citrate or (ethylenedinitrilo)tetraacetatoferrate (ferric EDTA) or mixtures thereof.

The compound obtained after the oxidation step is subjected to a subsequent reduction step. The reduction is performed by employing at least one peroxidase and/or heating.

Peroxidases are often heme containing enzymes, where heme is an iron-protoporphyrin IX that is capable to accept or donate electrons and to transit among the states of iron (II, III or IV). Any peroxidase which can perform the reduction of the compounds obtained in step ii) is suitable. Particularly suitable are peroxidases having the EC number horseradish peroxidase: 1.11.1.7, manganese peroxidase: 1.11.1.13, ascorbate oxidase: 1.10.3.3. The peroxidase used according to any aspect of the present invention may be selected from the group consisting of horseradish peroxidase, manganese peroxidase, salivary peroxidase, tryparedoxin peroxidase, heme peroxidase, ascorbate peroxidase or mixtures thereof. In particular, the peroxidase used according to any aspect of the present invention may be selected from the group consisting of horseradish peroxidase, manganese peroxidase and ascorbate oxidase. Even more in particular, the at least one peroxidase is horseradish peroxidase.

In one embodiment the at least one peroxidase is present in 0.01 to 1 wt.-%, based on the total weight of the at least one compound obtained in step ii). In another example, the peroxidase is present in 0.05 to 5, 0.1 to 5, 0.15 to 5, 0.2 to 5, 0.25 to 5, 0.3 to 5, 0.4 to 1, 0.5 to 5, 1 to 5, 1.5 to 5, 2 to 5, 2.5 to 5, 3 to 5, or 3.5 to 5 wt.-% based on the total weight of the at least one compound obtained in step ii. More in particular, the lipase is present in about 0.01, 0.5, 1, 1.5, 2, 2.5, 3, 3.5, 4, 4.5 or 5 wt.-% based on the total weight of the at least one compound obtained in step ii.

The adjustment of pH values is known to the skilled person by employing commonly known acids or bases.

In one embodiment the medium in which the steps are performed is an aqueous medium.

In one embodiment in step iii) at least one peroxidase and heating is employed, preferably at a temperature of 30 to 70° C. In particular, the reduction by peroxidase is carried out at 30-70, 35-70, 40-70, 45-70, 50-70, 55-70, 60-70, 30-66, 30-60, 30-55, 30-50, 30-45, 30-40° C. In one embodiment in step iii) the heating is performed at temperatures of 10 to 50° C., preferably 30 to 40° C., more preferably 40° C. If at least one peroxidase is present, preferably the temperature is at most 40° C., since the performance of the peroxidase might be influenced.

In another example, the temperature at which the reduction by peroxidase is carried out may be about 40, 45, 50, 55, 60, 65 or 70° C. Even more in particular, the temperature at which the reduction by peroxidase according to any aspect of the present invention is carried out may be about 70° C.

The reduction and oxidations steps can be performed in a single reaction vessel or in two different reaction vessels.

In one embodiment step iii) is performed 5 to 60, preferably 10 to 30, more preferably 15 to 25, minutes after adding the at least one lipoxygenase in step ii) or after 40 to 80 minutes after the start of the method according to any aspect of the present invention.

After the reduction step the pH value is adjusted to at most 4.5, preferably 3 to 4.5, more preferably 3.5.

The at least one compound obtained after step iv) according to any aspect of the present invention can be purified. Such purification steps are well known to a person skilled in the art, for example by centrifugation. In another example, purification of the compound obtained after step iv) may be carried out using for example, an adsorption column chromatography method using a carrier such as silica gel or alumina, an ion exchange chromatography method, or a normal-phase or reverse-phase column chromatography method using silica gel or alkylated silica gel (preferably, high performance liquid chromatography), or a normal-phase or reverse-phase column chromatography method using a filler, wherein an optically active molecule is fixed on the filler, or coated on silica gel (preferably, high performance liquid chromatography)). A skilled person would select the purification method that may be suitable based on the compound obtained after step iv).

In one embodiment the compound obtained after step iii) or iv) is 17-hydroxy docosahexaenoic acid (17-HDHA), 11-hydroxy docosahexaenoic acid (11-HDHA), 10-hydroxy docosahexaenoic acid (10-HDHA), 12-hydroxy eicosapentaenoic acid (12-HEPE), 15-hydroxy eicosapentaenoic acid (15-HEPE), 18-hydroxy eicosapentaenoic acid (18-HEPE), 5-hydroxyeicosatetraenoic acid (5-HETE), 11-hydroxyeicosatetraenoic acid (11-HETE), 12-hydroxyeicosatetraenoic acid (12-HETE), 4-hydroxy docosahexaenoic acid (4-HDHA), 7-hydroxy docosahexaenoic (7-HDHA) acid 13-hydroxy docosahexaenoic acid (13-HDHA), 14-hydroxy docosahexaenoic acid (14-HDHA), 20-hydroxy docosahexaenoic acid (20-HDHA), or 21-hydroxy docosahexaenoic acid (21-HDHA). In particular, the compound obtained after step iii) or iv) may be selected from the group consisting of 17-hydroxy docosahexaenoic acid (17-HDHA), 11-hydroxy docosahexaenoic acid (11-HDHA), 10-hydroxy docosahexaenoic acid (10-HDHA), 12-hydroxy eicosapentaenoic acid (12-HEPE), 15-hydroxy eicosapentaenoic acid (15-HEPE), 18-hydroxy eicosapentaenoic acid (18-HEPE), 5-hydroxyeicosatetraenoic acid (5-HETE), 11-hydroxyeicosatetraenoic acid (11-HETE), 12-hydroxyeicosatetraenoic acid (12-HETE), 4-hydroxy docosahexaenoic acid (4-HDHA), 7-hydroxy docosahexaenoic (7-HDHA) acid 13-hydroxy docosahexaenoic acid (13-HDHA), 14-hydroxy docosahexaenoic acid (14-HDHA), 20-hydroxy docosahexaenoic acid (20-HDHA), and 21-hydroxy docosahexaenoic acid (21-HDHA).

More in particular, the compound obtained after step iii) or iv) may be selected from the group consisting of 17-HDHA, 5-HETE, 11-HETE, 12-HETE, 15-HETE, 4-HDHA, 7-HDHA, 13-HDHA, 14-HDHA, 20-HDHA, 21-HDHA 12-HEPE, 15-HEPE, and 18-HEPE. These compounds may be produced according to any aspect of the present invention in a satisfying quantity.

### EXAMPLES

Methods and Materials

TABLE 1
Devices used in the Examples
Devices

| Equipment | Provider |
|---|---|
| DASGIP 1 L vessels | Eppendorf Vertrieb Deutschland |
| | GmbH, Germany |
| Centrifuge Sigma 4K15 | Sigma Laborzentrifugen GmbH, |
| | Germany |
| Magnetic Stirrer | IKA GmbH & Co. KG, Germany |
| Ice Machine | Zigra Eismaschinen GmbH, Germany |
| Thermomix | Vorwerk Deutschland Stiftung & Co. |
| | KG, Germany |
| Balance Kern EW 12000 | Kern & Sohn GmbH, Germany |
| Cryostat Minichiller 600 | Peter Huber Kältemaschinenbau AG, |
| | Germany |

TABLE 2
Materials used in the Examples
Materials

| Material | Supplier |
|---|---|
| AvailOm ® | Evonik Industries AG |
| KOH | Carl Roth GmbH + Co. KG |
| Soya Flour Davert | Mühle Schlingemann e.K. |
| Fresh Soya beans | Farm Hemmerde in (Waltrop) |
| Xiameter ™ ACP-1500 | Dow Inc. |
| Sulfuric acid 25% | Bernd Kraft GmbH |

### Example 1

Oxidation Step

Firstly, 50 g AvailOm® (that contains 7.5 g DHA) was dissolved in 262 g of deionized water in 1 L Eppendorf reactor. 0.2 mL of antifoam Xiameter™ ACP-150 were added to the solution to avoid foaming. The solution was homogenized using a stirrer (2×6-blade Rushton turbines) at 400 rpm for 1 hour at room temperature. The mixing of the DHA took place instantly to avoid large clumps, the final solution had a rosy color. The solution had a pH of 7.5, the pH of the solution was adjusted to 9.8 using 15 mL of 50% KOH. The Eppendorf reactor was cooled down to 5° C.

Preparation of the Soya Slurry:

Option 1 (OX1):

15 g of soya flour (DAVERT) was added to beaker glass that contains 50 g of water. The mixture was stirred using a magnetic stirrer in an ice bath for 30 minutes.

Option 2 (OX2):

15 g of fresh milled soya beans was added to beaker glass that contains 50 g of water. The mixture was stirred using a magnetic stirrer in an ice bath for 30 minutes.

5.13 g of the respective soya slurry were added to the DHA solution to start the oxidation step. The mixture was homogenized using a stirrer (2×6-blade Rushton turbines) at 400 rpm for 2 hours at 5° C. 3.2 g of the soya slurry was added to the mixture. The mixture was homogenized using the same parameters for another 2 hours. During the whole process the pH was held constant by the automized program of the Eppendorf reactor at 9.8 using the 50% KOH. The amount of the dissolved oxygen was measured continuously and controlled during the process to guarantee the enzyme had sufficient oxygen for the oxidation step. Increasing of stirrer speed and of the air flow rate was used for the supply of the oxygen. The decrease in the dissolved oxygen is an indication for the oxidation reaction.

After 4 hours the oxidation step was finished.

Reduction Step

Option 1 (RED 1):

To start the reduction, the temperature of the respective oxidized solution (OX1 or OX2) was respectively increased to 40° C. and the solution was flushed with nitrogen. pH of 9.5 was controlled by the addition of 50% KOH. Stirrer speed was adjusted to 400 rpm.

Option 2 (RED 2):

To start the reduction, 100 mg of horseradish peroxidase were added to the respective 100 ml oxidized solution (OX1 or OX2), respectively. The solution was incubated in 2×50 mL Falcon tubes in a shaker plate with 200 rpm for 1 h at room temperature.

After 2 h, the reduction step was finished by adding 12 mL of 2.5 M sulfuric acid to adjust pH to 3.5, respectively. Last step was phase separation by centrifugation. The solution was centrifuged in a Falcon Tube at 5000 g for 15 minutes, respectively. The upper phase was the oily product phase and the bottom phase is the aqueous one.

Samples were taken at the beginning and the end of each step and were analyzed by LC/MS.

HPLC Method for the Detection of DHA and 17-HDHA (the Method is Considered to be Qualitative)
Mobile Phase A Water+0.02% TFA (Trifluoroacetic Acid)
Mobile Phase B Acetonitrile+0.02% TFA

TABLE 3
HPLC conditions

| Time[min] | A [%] | B [%] | Flow [mL/min] |
|---|---|---|---|
| 0 | 85 | 15 | 0.6 |
| 1 | 85 | 15 | 0.6 |
| 9 | 2 | 98 | 0.6 |
| 12 | 2 | 98 | 0.6 |
| 12.1 | 85 | 15 | 0.6 |
| 17 | 85 | 15 | 0.6 |

| Column | Phenomenex Kinetex C18; 100 × 2.1 2.6 μm; | |
|---|---|---|
| | 100 A; Part No: 00D-4462-AN | |
| Oven Temperature | 60° | C. |
| Injection Volume | 1 | μL |
| Run Time | 17 | min |

| | |
|---|---|
| Detector | Triple Quad Mass Spectrometer |

TABLE 4
Detection of different compounds produced according
to any aspect of the present invention.

| | m/z | Mode |
|---|---|---|
| DHA | 351.2 | SIM |
| 17-HDHA | 367.1 | SIM |
| DiDHA | 325.1 | SIM |
| Linoleic Acid | 303.1 | SIM |
| 13-HPODE | 335.1 −> 317.1 | MRM |
| 13-HODE | 319.1 | SIM |

Results

HPLC Qualitative Results

The samples were measured by LC/MS and the quantification is calculated by a one-point calibration.

The results were shown in the tables below.

Reduction by Temperature Shift

Samples were taken from the experiment performing steps OX2 and RED1.

TABLE 5
DHA and 17-HDHA concentration from samples taken from
the experiment performing steps OX2 and RED1.

| | DHA | 17-HDHA |
|---|---|---|
| | concentration | concentration |
| | [mg/L] | [mg/L] |
| start of oxidation step | 22919 | 41 |
| end of oxidation step | 21641 | 246 |
| start of reduction step (by | 20811 | 249 |
| temperature shift) | | |
| end of reduction step (by | 19981 | 537 |
| temperature shift) | | |
| upper phase after centrifugation | 39608 | 851 |
| bottom phase after centrifugation | 25 | 1 |

In addition to 17-HDHA following compounds were obtained in the experiment in sufficient amounts as well: 5-HETE, 11-HETE, 12-HETE, 15-HETE, 4-HDHA, 7-HDHA, 13-HDHA, 14-HDHA, 20-HDHA, 21-HDHA 12-HEPE, 15-HEPE, and 18-HEPE.

Reduction by Horseradish Peroxidase

Samples were taken from the experiment performing steps OX2 and RED2.

TABLE 6
DHA and 17-HDHA concentration from samples taken from
the experiment performing steps OX2 and RED2.

| | DHA | 17-HDHA |
|---|---|---|
| | concentration | concentration |
| | [mg/L] | [mg/L] |
| start of oxidation step | 20977 | 24 |
| end of oxidation step | 19291 | 294 |
| start of reduction step (by | 19795 | 320 |
| temperature shift) | | |
| end of reduction step (by | 10637 | 478 |
| temperature shift) | | |
| upper phase after centrifugation | 24960 | 1969 |
| bottom phase after centrifugation | 1 | 1 |

In addition to 17-HDHA following compounds were obtained in the experiment in sufficient amounts as well: 5-HETE, 11-HETE, 12-HETE, 15-HETE, 4-HDHA, 7-HDHA, 13-HDHA, 14-HDHA, 20-HDHA, 21-HDHA 12-HEPE, 15-HEPE, and 18-HEPE.

Similar results were obtained for the reactions OX1+RED1 and OX1+RED2.

### Example 2

Reduction Step

Option 1 (RED 3):

To start the reduction, the temperature of the respective oxidized solution (OX1 or OX2) was respectively increased to 70° C. and the solution was flushed with nitrogen. pH of 9.5 was controlled by the addition of 50% KOH. Stirrer speed was adjusted to 400 rpm.

The method used is as described in Example 1. Samples were taken at the beginning and the end of each step and were analyzed by LC/MS.

Reduction by Temperature Shift

Samples were taken from the experiment performing steps OX2 and RED3.

TABLE 7
DHA and 17-HDHA concentration from samples taken from
the experiment performing steps OX2 and RED3.

| | DHA | 17-HDHA |
|---|---|---|
| | concentration | concentration |
| | [mg/L] | [mg/L] |
| start of oxidation step | 19710 | 1630 |
| end of oxidation step | 51430 | 5840 |
| start of reduction step (by | 57100 | 24160 |
| temperature shift) | | |
| end of reduction step (by | 52290 | 49200 |
| temperature shift) | | |

In addition to 17-HDHA following compounds were obtained in the experiment in sufficient amounts as well: 5-HETE, 11-HETE, 12-HETE, 15-HETE, 4-HDHA, 7-HDHA, 13-HDHA, 14-HDHA, 20-HDHA, 21-HDHA 12-HEPE, 15-HEPE, and 18-HEPE.

### Example 3

Testing Different Oxidation Temperatures
Option 1 (TEMP1): 5° C. oxidation temperature
Option 2 (TEMP2): 10° C. oxidation temperature
Option 3 (TEMP3): 15° C. oxidation temperature

Oxidation was carried out as disclosed in Example 1.

Samples were taken from the experiment performing steps OX2 and TEMP1.

TABLE 8
DHA and 17-HDHA concentration from samples taken from
the experiment performing steps OX2 and TEMP1.

| | DHA concentration | 17-HDHA concentration |
|---|---|---|
| | [mg/L] | [mg/L] |
| start of oxidation step | 118720 | 4810 |
| end of oxidation step | 112030 | 49690 |

Samples were taken from the experiment performing steps OX2 and TEMP2.

TABLE 9
DHA and 17-HDHA concentration from samples taken from
the experiment performing steps OX2 and TEMP2.

| | DHA concentration | 17-HDHA concentration |
|---|---|---|
| | [mg/L] | [mg/L] |
| start of oxidation step | 122610 | 5120 |
| end of oxidation step | 96580 | 47620 |

Samples were taken from the experiment performing steps OX2 and TEMP3.

TABLE 10
DHA and 17-HDHA concentration from samples taken from
the experiment performing steps OX2 and TEMP3.

| | DHA concentration | 17-HDHA concentration |
|---|---|---|
| | [mg/L] | [mg/L] |
| start of oxidation step | 133920 | 2630 |
| end of oxidation step | 96180 | 28180 |

In addition to 17-HDHA following compounds were obtained in the experiment in sufficient amounts as well: 5-HETE, 11-HETE, 12-HETE, 15-HETE, 4-HDHA, 7-HDHA, 13-HDHA, 14-HDHA, 20-HDHA, 21-HDHA 12-HEPE, 15-HEPE, and 18-HEPE.

1. A method for producing hydroxylated fatty acids, comprising the steps:
i) optionally saponification or hydrolyzation of at least one unsaturated fatty acid ester to obtain at least one unsaturated fatty acid;
ii) oxidizing the at least one unsaturated fatty acid, by at least one lipoxygenase, wherein the oxidizing is performed at temperatures of 5 to 10° C.;
iii) reducing the at least one compound obtained in step ii) by
iiia) at least one peroxidase, and/or
iiib) heating, and thereafter
iv) adjusting the pH value to at most 4.5 to obtain at least one hydroxylated fatty acid; and
v) optionally purifying the at least one compound obtained in step iv).

2. The method according to claim 1, wherein the at least one unsaturated fatty acid is at least one omega-3 fatty acid.

3. The method according to claim 2, wherein the omega-3 fatty acid is selected from the group consisting of docosahexanoic acid, eicosapentaenoic acid and a mixture thereof.

4. The method according to claim 1, wherein the oxidizing step (ii) is performed at about 5° C.

5. The method according to claim 1, wherein the at least one lipoxygenase is obtained from soy.

6. The method according to claim 1, wherein in step ii) in addition to the at least one lipoxygenase, at least one co-factor is present.

7. The method according to claim 1, wherein the at least one peroxidase is selected from the group consisting of horseradish peroxidase, manganese peroxidase, salivary peroxidase, tryparedoxin peroxidase, heme peroxidase, ascorbate peroxidase and mixtures thereof.

8. The method according to claim 1, wherein the heating is performed at a temperature of 10 to 70° C.

9. The method according to claim 1, wherein the heating is performed at a temperature of about 70° C.

10. The method according to claim 1, wherein the compound obtained after step iv) is selected from the group consisting of 17-hydroxy docosahexaenoic acid, 11-hydroxy docosahexaenoic acid, 10-hydroxy docosahexaenoic acid, 12-hydroxy eicosapentaenoic acid, 15-hydroxy eicosapentaenoic acid, 18-hydroxy eicosapentaenoic acid, 5-hydroxyeicosatetraenoic acid, 11-hydroxyeicosatetraenoic acid, 12-hydroxyeicosatetraenoic acid, 4-hydroxy docosahexaenoic acid, 7-hydroxy docosahexaenoic acid 13-hydroxy docosahexaenoic acid, 14-hydroxy docosahexaenoic acid, 15-hydroxyeicosatetraenoic acid, 20-hydroxy docosahexaenoic acid, and 21-hydroxy docosahexaenoic acid and any mixture thereof.

11. The method according to claim 1, wherein steps ii) and iii) are performed in a single reaction vessel or in two different reaction vessels.

12. The method according to claim 1, wherein step iii) is performed 5 to 60 minutes after completely adding the at least one lipoxygenase in step ii) or after 40 to 80 minutes after the start of the method.

13. Compound obtained by the method according to claim 1.
